(19) United States
(12) Patent Application Publication
(10) Pub. No.: US 2004/0115288 A1
(43) Pub. Date: Jun. 17, 2004

(54) Method for producing vitamin b enriched preparations from grasses

(76) Inventors: Behzad Sadeghi, Wien (AT); Peter Kössler, Wien (AT); Norbert Fuchs, Mariapffar (AT)

Correspondence Address: FULBRIGHT & JAWORSKI L.L.P., 600 CONGRESS AVE., SUITE 2400, AUSTIN, TX, 78701, US

(21) Appl. No.: 10/472,368

(22) PCT Filed: Mar. 19, 2002
(86) PCT No.: PCT/AT02/00089

(30) Foreign Application Priority Data
Mar. 21, 2001 (AT) A4502001

(51) Int. Cl. A61K 35/78

(52) U.S. Cl. 424/750; 47/58.1 R

(57) ABSTRACT

A method for the preparation of vitamin B enriched preparations made from plants is described, characterized in that plants are grown until prior to the budding phase at the longest, said plants are cut, said plants are left to grow again, said plants are cut again, with said second cut being collected, said step of leaving the grass to grow again and cutting it again being optionally repeated at least once, and said further cuts being collected and combined, and said second cut or said second and further cuts being processed to a vitamin B enriched plant preparation.

The invention relates to a method for the preparation of vitamin B enriched preparations made from plants.

Vitamins of the B complex are applied in various ways in the fields of the pharmaceutical industry and in the food industry. While, however, for certain vitamins (e.g. vitamin C, vitamin E) a variety of natural resources for the preparation of vitamin C enriched preparations is available (e.g. acerola cherries, rose hip fruits, oily extracts of wheat germs and other grains for natural vitamin E resources), vitamins of the B complex are mainly prepared synthetically and/or microbiologically. However, such synthetic or microbiological preparation methods are not without problems, particularly with respect to contaminations with irritating substances which get into the preparation in the course of the synthesis pathway. Moreover, the vitamins of the B complex, e.g. when prepared by (manipulated) microorganisms, are provided in combination with substances with which they are not naturally associated, e.g. in plants.

Furthermore, essential trace elements, such as e.g. selenium or molybdenum, are often used for animals, humans, and plants in inorganic or organic-synthetic form in pharmaceutical or food technology preparations.

However, what is desired for both vitamin B preparations and preparations containing essential trace elements is that such preparations be provided in efficient form, preferably in combination with the natural metabolic partners with which they are present in plant organisms.

SU 1 825 294 A3 relates to mixtures of hedysarum (esparsette) or Sudan grass with corn (maize), mentioning two mowing phases, i.e. one at 175 to 190 cm and another at 135 to 140 cm. The object of this procedure is to obtain protein rich feed grass by special seeding and mowing conditions. Said document does not mention the micronutrient content, such as e.g. B-vitamins or trace elements.

According to SU 1 727 600 A1, a step of preliminary mowing of plants prior to the budding phase at a height of about 12 to 18 cm is provided. The only object of this procedure is to increase grass or fruit yield per given area of cultivation by mowing twice. Said document neither describes nor implies influencing the vitamin content by repeated mowing.

According to RU 2 108 731 C a plant feed material is prepared, with coagulation agents and preservatives being added. Said document only describes mechanical and chemical methods for the production of nutrient rich animal feeds.

U.S. Pat. No. 5,773,681 A relates to the preparation of electrolyte enriched germs. Said document describes a method for enriching germinable seeds with electrolytes without putting them into soil. Said document does not contain any information on the mowing strategy of plants or on the vitamin B content of plants in relation to certain cutting strategies.

EP 0 652 707 B1 relates to the preparation of encrusted seeds, the primary object being the application of nutrients such as nitrogen, phosphorous, potassium and/or microelements onto seeds using chitosan (a natural polymerisate of sea crustaceae).

None of these known methods, however, looks into the vitamin B content of the plants nor attempts to raise the vitamin B content.

Therefore, the object of the present invention is the development of a method and the application of techniques to obtain highly concentrated vitamin B complexes from natural resources. These methods should be industrially applicable in standardizable and reproducible form. Moreover, said vitamin B complex preparations obtained from natural resources should preferably be improved with respect to their content of inorganic trace element compounds, which should be organically available in the final preparation.

Accordingly, the subject matter of the present invention, which realizes one or more of said objects, is a method for the preparation of vitamin B enriched preparations from plants, wherein plants are grown until prior to the budding phase at the longest, said plants are cut, said plants are left to grow again, said plants are cut again, with said second cut being collected, said step of leaving the plants to grow again and cutting them again being optionally repeated at least once, and said further cuts being collected and combined, and said second cut or said second and further cuts being processed to a vitamin B enriched plant preparation.

What is essential is that prior to the first cut the plants only grow as high as to reach a pre-budding stage and the first cut is effected at this point at the latest. This is important, because the plants are not able to shoot up again for a second or even third time after they have reached the budding stage. As a rule, in wheat grasses said height of growth (shortly prior to budding) is about 20-30 cm. Other grasses or plants to be used according to the present invention may reach lower (lucerne) or higher heights of growth before they reach the budding stage.

Surprisingly, according to the invention it was found that the vitamin content, particularly the vitamin B1 and vitamin B2 content, was significantly higher in the second and further cuts of plants than in the first cut and that thus vitamin B preparations may be obtained which are extremely rich in B-vitamins. Moreover, the B-vitamins in the preparations prepared according to the present invention are present in combination with numerous natural plant interaction partners; of course, there are no contaminations whatsoever which could have got into the preparation during chemical synthesis or microbiological processes (catalyst residues, recombinant, microbial or other genetically foreign material, . . . ).

Preferably, the method according to the present invention is carried out using plants which are grown out of electrolyte encrusted seeds. For encrusting, the seeds are treated with a fertilizer solution containing essential trace elements, such as e.g. selenium, molybdenum, chromium, manganese, copper, ammonium, or zinc. Preferably, the solution is sprayed onto the surface of the seeds and fixed on the surface by means of binding agents.

Moreover, electrolytically enriched germs, such as described in e.g. EP 0 770 324 A2, may also be preferably used as starting materials for said plants.

The plants thus grown are richer in trace elements, such as chromium, manganese, iron, copper, selenium, molybdenum, etc., than seeds or germs incubated with the usual tap or rain water.

Preferably, said electrolyte encrusted seeds are obtained by treating seeds with an encrusting solution containing sodium selenate, sodium molybdate, chromium-III-chloride, manganese chloride, copper gluconate, ammonium-iron-III-citrate, zinc gluconate or mixtures thereof, with the concentration of said salts being preferably selected to be in the range of between 0.1 mg-10 g/200 g of encrusting solution. Preferably, the concentration, relative to 200 g of encrusting solution, of sodium selenate is 0.1 to 100 mg, particularly 1 to 50 mg, of sodium molybdate is 0.1 to 100 mg, particularly 1 to 50 mg, of chromium-III-chloride is 0.5 to 500 mg, particularly 1 to 100 mg, of manganese chloride is 1 to 1000 mg, particularly 10 to 500 mg, of copper gluconate is 1 to 2000 mg, particularly 10 to 1000 mg, of ammonium-iron-III-citrate is 1 to 2000 mg, particularly 10 to 1000 mg, of copper gluconate is 1 to 2000 mg, particularly 10 to 1000 mg, and of zinc gluconate is 1 to 2000 mg, particularly 10 to 1000 mg.

The present invention is useful for raising the vitamin B content in all vitamin B containing plants. Preferably, leguminous plants and grain seeds are treated according to the present invention. Particularly preferred are the plants of wheat, buckwheat, quinoa, mungo beans, fenugreek, radish, alfalfa, corn (maize), pumpkin, rye, barley, rice, azuki beans, peas, millet, chick-peas, cress, linseed, lentils, mustard, sesame, soy beans, sunflowers, and amaranth, particularly wheat.

Preferably, the vitamin B enriched plant preparation obtained is processed to plant juice or plant juice powder, which may then be used further mainly in the field of pharmaceuticals or food.

Preferably, the plants are first cut at a height of about 20 to 30 cm (because at this height of growth most of the commercially useful plants (grasses, for example wheat grasses) have not yet started budding), and preferably the plants are cut at a distance of less than 15 cm, particularly less than 10 cm from the soil (on average). The further growth steps may then also be preferably up to a height of more than 20 cm, and the further cuts may be carried out analogously to the first cut. The cut plants may then be pressed to juice and/or spray dried and may then be stored for a longer period of time mainly in the form of plant juice powder. The press residue (press cake) also represents a vitamin B enriched plant preparation within the scope of the present invention, which has an increased vitamin B content.

Another subject matter of the present invention is a vitamin B enriched plant preparation obtainable according to the method of the present invention. This preparation may then serve as the basis (natural raw material) for further use particularly in pharmaceutical and food preparations. The preparations according to the present invention, particularly the vitamin B rich plant juice extracts, may be used in the pharmaceutical field as tablets, capsules, powder mixtures to be dissolved, effervescent tablets, ready-made solutions, oral and external emulsions and suspensions, injectables, suppositories, transdermal systems, veterinary products and hospital food. In the field of food, the preparations obtainable according to the present invention may be provided in cereals, all kinds of milk products, instant meals, all kinds of liquid preparations, feeds for veterinary use, mueslis and muesli bars, energy bars, sports nutrition, nutrition for the elderly, instant formulations, paste-like formulations, spaghetti, pasta and other noodle dishes and baking mixtures; they are particularly useful because they are enriched in vitamin B and optionally trace elements and will help improve the general state of health of the consumer of these preparations.

The invention will be explained in greater detail using the following examples, to which it is not limited, of course.

### EXAMPLE 1

Wheat seeds were encrusted with fertilizer solutions of the following composition:

Composition in mg/200 g of encrusting solution:

TABLE 1

| | Concentration | Concentration | Concentration |
|---|---|---|---|
| | 1-fold | 5-fold | 10-fold |
| Sodium selenate | 1.625 mg | 8.125 mg | 16.25 mg |
| Sodium | 1.260 mg | 6.300 mg | 12.60 mg |
| molybdate | | | |
| Chromium-III- | 5.120 mg | 25.600 mg | 51.20 mg |
| chloride | | | |
| Manganese | 14.750 mg | 73.750 mg | 147.50 mg |
| chloride | | | |
| Copper | 35.700 mg | 178.500 mg | 357.00 mg |
| gluconate | | | |
| Ammonium-iron- | 35.700 mg | 178.500 mg | 357.00 mg |
| III-citrate | | | |
| Zinc gluconate | 78.00 mg | 394.000 mg | 788.00 mg |

1000 g of wheat seeds (type Dekan untreated (company SAREA-Samenpille)) each were encrusted with 200 g of the fertilizer solution mentioned above, i.e. the solution was sprayed onto the surface of the wheat seeds and fixed on the surface by means of a binding agent. Subsequently, the three batches of wheat seeds, each sprayed upon with different intensity, and a sample of the wheat on which no trace element solution had been sprayed upon were put into the soil (429 g/m², i.e. 199 g/0.46 m²).

Subsequently, the 4 experimental fields were sprayed with conventional tap water according to the usual cultivating methods for about 4 weeks until the wheat grass reached a height of about 25 cm. Then the wheat grass was cut at a distance of about 3 cm from the soil, pressed to juice according to the same conditions and spray dried. Subsequently, the dry wheat grass juice powder (WGJP) and the comparative sample (wheat, free field cultivation) were analyzed for their trace element content by means of ICP-MS. For comparative purposes, the press residues (press cakes) were also analyzed for their trace element content. The results are given in table 2 below.

TABLE 2

| | WGJP 1 | WGJP 5 | WGJP 10 |
|---|---|---|---|
| | encrusted | encrusted | encrusted |
| Cr | 1.64 ± 0.08 | 1.81 ± 0.10 | 1.74 ± 0.04 |
| Mn | 106 ± 3 | 108 ± 6 | 104 ± 1 |
| Fe | 197 ± 6 | 200 ± 8 | 229 ± 1 |
| Cu | 20.5 ± 0.1 | 19.4 ± 0.6 | 21.2 ± 0.1 |
| Zn | 76 ± 1 | 70 ± 1 | 75 ± 2 |
| Se | 4.13 ± 0.05 | 19.5 ± 0.9 | 45.2 ± 0.3 |
| Mo | 12.9 ± 0.2 | 19.3 ± 0.4 | 26.5 ± 0.2 |
| | Press cake 1 | Press cake 5 | Press cake 10 |
| | encrusted | encrusted | encrusted |
| Cr | 1.0 ± 0.2 | 0.6 ± 0.4 | 0.9 ± 0.3 |
| Mn | 43 ± 2 | 43 ± 4 | 44 ± 5 |
| Fe | 93 ± 5 | 85 ± 10 | 94 ± 3 |
| Cu | 7.1 ± 0.8 | 5.8 ± 0.8 | 6.6 ± 0.3 |
| Zn | 37 ± 3 | 39.7 ± 0.9 | 45 ± 5 |
| Se | 1.1 ± 0.1 | 3.8 ± 0.4 | 8.3 ± 0.7 |
| Mo | 8.1 ± 0.2 | 10 ± 2 | 13 ± 1 |

| | Wheat free field cultivation 0045 |
|---|---|
| Cr | 0.58 ± 0.07 |
| Mn | 38 ± 1 |
| Fe | 57 ± 4 |
| Cu | 6.0 ± 0.2 |
| Zn | 33 ± 2 |
| Se | 0.36 ± 0.04 |
| Mo | 0.62 ± 0.01 |

All results in mg/kg (n=3).

The analyses of the vitamin B content of wheat grass juice powders from wheat seeds which were once enriched with trace element solution but not subjected to preliminary germination or encrusted with fertilizer solution revealed no significant influences on the content of B-vitamins, particularly vitamin B1 and vitamin B2, in this first cut.

In the present example, after 1 to 2 weeks wheat grass grew again from the seeds which had remained in the soil, and it was cut again (2^{nd} cut). If this 2^{nd} cut is harvested, too, the wheat grass will grow for a third time from the seeds which have remained in the soil (3^{rd} cut). An analysis of the three wheat grass cuts revealed a significant rise in the content of B-vitamins from the first to the third cut, as shown in table 3.

TABLE 3

| | Riboflavin content |
|---|---|
| Products | in mg/kg |
| Wheat Capo | 0.7 |
| PMN (Stamag) 4.5 days germinated | 2.2 |
| PMN leaf (8.5 days) | 12 |
| Wheat grass juice from climate chamber | 1.8-1.9 |
| Wheat grass juice from field trial (1^{st} | 1.9-2.2 |
| cut) | |
| Wheat grass juice from field trial (2^{nd} | 2.8-3.2 |
| cut) | |
| Wheat grass juice from field trial (3^{rd} | 4.1-4.4 |
| cut) | |
| Wheat grass juice powder from climate | 28.8-30.4 |
| chamber | |
| Wheat grass juice powder (1^{st} cut) | 30.4-35.2 |
| Wheat grass juice powder (2^{nd} cut) | 44.8-51.2 |
| Wheat grass juice powder (3^{rd} cut) | 65.6-70.4 |
| PMN germ | 7.0-7.5 |
| PMN bran | 6.0-6.5 |

These increased contents of B-vitamins, which have not been described before in the literature, show that it is possible to stimulate the production of vitamin B in the growing plants by repeatedly harvesting plants, particularly wheat grasses, without any additional means. By means of subsequent technological processes (centrifugation, nanofiltration, spray drying, etc.), wheat grass thus obtained, being high in B-vitamins, may result in natural raw materials having far higher contents of naturally grown B-vitamins than conventional materials.

What we claim is:

1. A method for the preparation of vitamin B enriched preparations made from grasses, characterized in that grasses are grown until prior to the budding phase at the longest, said grasses are cut, said grasses are left to grow again, said grasses are cut again, with said second cut being collected, said step of leaving the grass to grow again and cutting it again being optionally repeated at least once, and said further cuts being collected and combined, and said second cut or said second and further cuts being processed to a vitamin B enriched grass preparation.

2. A method according to claim 1, characterized in that said grasses are grown out of electrolyte encrusted seeds.

3. A method according to claim 2, characterized in that said electrolyte encrusted seeds are obtained by treating seeds with an encrusting solution containing sodium selenate, sodium molybdate, chromium-III-chloride, manganese chloride, copper gluconate, ammonium-iron-III-citrate, zink gluconate or mixtures thereof.

4. A method according to claim 3, characterized in that said encrusting solution contains sodium selenate, sodium molybdate, chromium-III-chloride, manganese chloride, copper gluconate, ammonium-iron-III-citrate or zink gluconate at a concentration of 0.1 mg-100 g/200 g of encrusting solution.

5. A method according to claims 3 or 4, characterized in that said encrusting solution contains, relative to 200 g of encrusting solution, sodium selenate at a concentration of from 0.1 to 100 mg, particularly 1 to 50 mg, sodium molybdate at 0.1 to 100 mg, particularly 1 to 50 mg, chromium-III-chloride at 0.5 to 500 mg, particularly 1 to 100 mg, manganese chloride at 1 to 1000 mg, particularly 10 to 500 mg, copper gluconate at 1 to 2000 mg, particularly 10 to 1000 mg, ammonium-iron-III-citrate at 1 to 2000 mg, particularly 10 to 1000 mg, copper gluconate at 1 to 2000 mg, particularly 10 to 1000 mg, and zink gluconate at 1 to 2000 mg, particularly 10 to 1000 mg, or mixtures thereof.

6. A method according to any one of claims 1 to 5, characterized in that the grasses of leguminous plants and grains are used.

7. A method according to any one of claims 1 to 6, characterized in that the grasses of wheat, buckwheat, quinoa, mungo beans, fenugreek, radish, alfalfa, corn (maize), pumpkin, rye, barley, rice, azuki beans, peas, millet, chick-peas, cress, linseed, lentils, mustard, sesame, soy beans, sunflowers, and amaranth, particularly wheat grasses are used.

8. A method according to any one of claims 1 to 7, characterized in that said second cut or said second and further cuts are processed to a vitamin B enriched grass juice or grass juice powder.

9. A vitamin B enriched grass preparation obtainable according to any one of claims 1 to 8.

10. A pharmaceutical composition containing a grass juice preparation according to claim 9.

11. A food composition containing a vitamin B enriched grass preparation according to claim 9.
